(19) United States
(12) Patent Application Publication
(10) Pub. No.: US 2026/0030378 A1
(43) Pub. Date: Jan. 29, 2026

(54) ARTIFICIAL INTELLIGENCE BASED SERVICE COLLABORATION

(71) Applicant: Ocean Deep Technologies LLC, Jacksonville, FL (US)

(72) Inventor: Saima A. Gonsalves, Victoria, MN (US)

(73) Assignee: Ocean Deep Technologies LLC, Jacksonville, FL (US)

(21) Appl. No.: 19/260,261

(22) Filed: Jul. 3, 2025

Related U.S. Application Data
(60) Provisional application No. 63/675,222, filed on Jul. 24, 2024.

(51) Int. Cl. G06F 21/62; G06F 21/60; G06Q 10/1093; G16H 10/60

(52) U.S. Cl. CPC G06F 21/6218; G06F 21/602; G06Q 10/1093; G16H 10/60

(57) ABSTRACT

AI based ubiquitous online consultations method and system is disclosed. A computer-implemented method comprises receiving an electronic input from one or more service providers, wherein the electronic input includes a set of credentials associated with the one or more service providers worldwide. The method further comprises verifying the one or more service providers based on the set of credentials that are validated from one or more regulatory authorities including licensing authority, certification authority, and professional association; and preparing a database of service providers by including the verified one or more service providers. The method further comprises implementing an access control mechanism based on a user's role, permission, and authentication credentials to prevent unauthorized access from viewing or modifying the database. The method further comprises employing an encryption protocol to encrypt the database at rest and in transit.

### CLAIM FOR PRIORITY

This application claims priority to U.S. Provisional Patent Application No. 63/675,222, filed on Jul. 24, 2024, titled “ARTIFICIAL INTELLIGENCE BASED SERVICE COLLABORATION,” which is incorporated by reference in its entirety for all purposes. Each of these applications is hereby incorporated by reference in its entirety for all purposes.

### BACKGROUND

Clients in various regions or countries of the world may not have access to competent and certified service providers. Clients may find it difficult to reach service providers, using mobile, broadband, or similar communication networks, for resolution of problems faced by the clients, for example, repair of electric appliances, access to health care, maintenance of automobiles, preparing tax returns, etc. To get access to ubiquitous consultations by service providers across different geographical jurisdictions, ensuring security and privacy of clients' data may become a significant concern depending on the nature of services such as tax return filing service where security of financial data might become a significant concern.

The background description provided here is for the purpose of generally presenting the context of the disclosure. Unless otherwise indicated here, the material described in this section is not prior art to the claims in this application and is not admitted to be prior art by inclusion in this section.

### BRIEF DESCRIPTION OF DRAWINGS

The examples will be understood more fully from the detailed description given below and from the accompanying drawings, which, however, should not be taken to limit the disclosure to the specific examples, but are for explanation and understanding only.

FIG. 1 illustrates a world map with a client in one jurisdiction consulting a service provider in a different jurisdiction of the world, in accordance with at least one embodiment.

FIG. 2 illustrates an AI based ubiquitous consultation session between a service provider and a client, in accordance with at least one embodiment.

FIG. 3 illustrates a computerized method and associated hardware of client initiating consultation with a service provider, in accordance with at least one embodiment.

FIG. 4 illustrates a secure method and associated hardware of registration and approval of one or more service providers, in accordance with at least one embodiment.

FIG. 5 illustrates an input form to be filled in by a client, in accordance with at least one embodiment.

FIG. 6 illustrates a method and associated hardware of preparing consolidated medical record of a subject, in accordance with at least one embodiment.

FIG. 7 illustrates a computer architecture for secure electronic session for consultation between client and service providers, in accordance with at least one embodiment.

FIG. 8 illustrates a web application process and associated hardware to sign into a system application programming interface (API) server, in accordance with at least one embodiment.

FIG. 9 illustrates a system and associated workflow that enables a user to use resources of online consultation services platform.

FIG. 10 illustrates a method of data encryption and associated hardware, in accordance with at least one embodiment.

FIG. 11 illustrates a computer system to implement one or more methods of consultation between a service provider and a client, in accordance with at least one embodiment.

FIG. 12 illustrates a computer system having graphical processing units (GPUs) for execution of various machine learning methods, in accordance with at least one embodiment.

### DETAILED DESCRIPTION

Some services may be provided remotely to clients, using a communication link, by service providers who are not even resident in the same country. Language and cultural diversity can make it difficult to better understand a problem from the context of conversation. A lack of mastery of a language, coupled with the inherent ambiguity in the natural language, might hamper seamless and effective communication between clients and service providers. Culture defines the social contract in a society that lays the foundation of business ethics in a population. Business ethics sets a bar for quality, reliability, and cost of services that clients expect from service providers. In at least one embodiment, to have access to a professional, reliable, and cost-efficient service, provided by service provider residing in a different jurisdiction, a user may use artificial intelligence (AI) based language translation services in real time so that competent service providers can better understand the clients' issues and resolve them efficiently and effectively to the satisfaction of clients. In at least one embodiment, AI assisted sign language communication is provided to address cases where a client/user is deaf or has special needs and needs to communicate with a service provider (e.g., practitioner). In at least one embodiment, an AI assisted sign language module converts service provider's speech to sign language for the client and client's sign language is communicated to service provider into speech/language giving the experience of normal verbal communication.

Some examples relate to AI based ubiquitous consultation services that may be remotely provided. A database of one or more service providers on a global level having one or more type of service providers may be created. Clients may contact the service providers and discuss one or more problems to get expert opinions for one or more solutions from one or more service providers. In at least one embodiment, a database of one or more service providers is generated. Service providers from anywhere in the world may be invited to register with the service providers' database by entering personal and professional credentials through an interface. The one or more service providers may be verified by validating the set of professional credentials from one or more online databases of relevant regulatory bodies or authorities. Verified service providers may be registered as certified service providers and stored in the database. To explain various examples, service providers are considered as health care service providers and clients are patients. However, the methods of AI based ubiquitous consultation services can be used for any service provider and corresponding clients.

In terms of healthcare services, it is possible that a subject might have siloed electronic health records that are stored with multiple electronic health record (EHR) providers; as a result, a healthcare provider may not be able to access these records and then aggregate them to have a better understanding of the digital health state of a subject at the time of consultation. This problem is aggregative, if a subject belongs to a country where it is not a requirement to maintain electronic healthcare records in a standardized format in an EHR system. A healthcare provider might ask for the healthcare records of a subject to better understand his health profile, and in that case the subject might have to obtain paper-based healthcare records from different providers, scan and aggregate them, and then send them to the provider. This process might take a significant amount of time and can lead to delays in correct diagnosis, which can further delay the start of the treatment.

To facilitate clients in different geographical jurisdictions, especially in regions having limited access to skilled service providers, some examples provide techniques to the clients to ubiquitously find and consult service providers. Clients all over the world may not have knowledge and access to appropriate service providers who are competent and certified to solve the problems of clients. At least one example provides a database of verified and certified service providers across one or more jurisdictions. In at least one embodiment, professional credentials associated with one or more service providers are received. In at least one embodiment, the credentials provided by one or more service providers are verified in online databases of relevant regulatory bodies or authorities or professional associations constituted by following due process of law. In at least one embodiment, one or more verified service providers are stored in the database.

In at least one embodiment, a client registers for a remote consultation, and provides an input describing the client's problem in a natural language format, such as audio or text. In at least one embodiment, by leveraging one or more machine-learning (ML) methods, the problem is identified from parsing the description.

In at least one embodiment, a client is a subject and a service provider is a healthcare provider. In various jurisdictions, the subject may not have access to information networks including internet for accessing electronic health records. In at least one embodiment, the system pulls one or more electronic health records of the subject from one or more electronic health record EHR providers. In at least one embodiment, the subject may upload one or more records from the local computer memory of the subject. In at least one embodiment, the subject provides additional records on the request of a provider. Based on the uploaded electronic healthcare records, the system may aggregate them into one record so that a provider may better comprehend the history healthcare states and associated management interventions by earlier providers. In at least one embodiment, the subject is quizzed by an AI system to infer the subject's likely digital health state and then present him a likely list of chronic conditions he might be suffering from. The subject may select one or more conditions based on the current health state of the subject. In at least one embodiment, based on the healthcare conditions selected by the subject, a list of one or more certified healthcare providers, matching the subject's preference criteria by leverage AI based semantic matching methods, is retrieved and shown to the subject on the electronic screen of subject's device. In at least one embodiment, the subject selects a provider from the list. In at least one embodiment, an input form is prepared that includes different queries related to the subject: for example, smoking history, alcohol consumption patterns, dietary restrictions, any allergies to medicine or food, etc. In at least one embodiment, the input form and the record of the subject are shared with the selected provider.

In at least one embodiment, an appointment may be scheduled between the subject and the selected provider by using AI based digital assistants, which can dynamically review the calendar of the provider and identify the available free time slots of the provider and display them to the subject from which the subject can reserve a suitable slot. At the scheduled time, a consultation session between the subject and the selected provider may be established through a video conferencing application. The video conferencing application can be Zoom, Microsoft Teams, FaceTime, Google Meets, or any other communication method. In at least one embodiment, an automated transcription of the conversations during the consultation session is generated. In a global ubiquitous consultation session, subjects and providers may speak different languages; therefore, In at least one embodiment, automated translation between the languages in real-time can be provided. In at least one embodiment, the transcription of the conversation during the consultation session is processed by an AI based natural language processing model and certain phenotypes, biomarkers, and relevant information elements, that together may determine the digital health state of a patient, along with the patient management interventions recommended, are extracted. These information elements are then aggregated with the multimodal information stored in the electronic medical records of the subject to determine the digital health state of a patient and then generative AI based models can be used to generated accurate provider notes in a desired natural language. In at least one embodiment, the provider notes are generated for the provider using the medicine terms and by following the writing style of a provider. In at least one embodiment, a simplified note using the everyday language of a common man is generated for the subject so that the subject can better understand the information in the note. In at least one embodiment, a provider may request to also generate a summary of the detailed note and store in the EHR system for a quick reference for future consultations.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

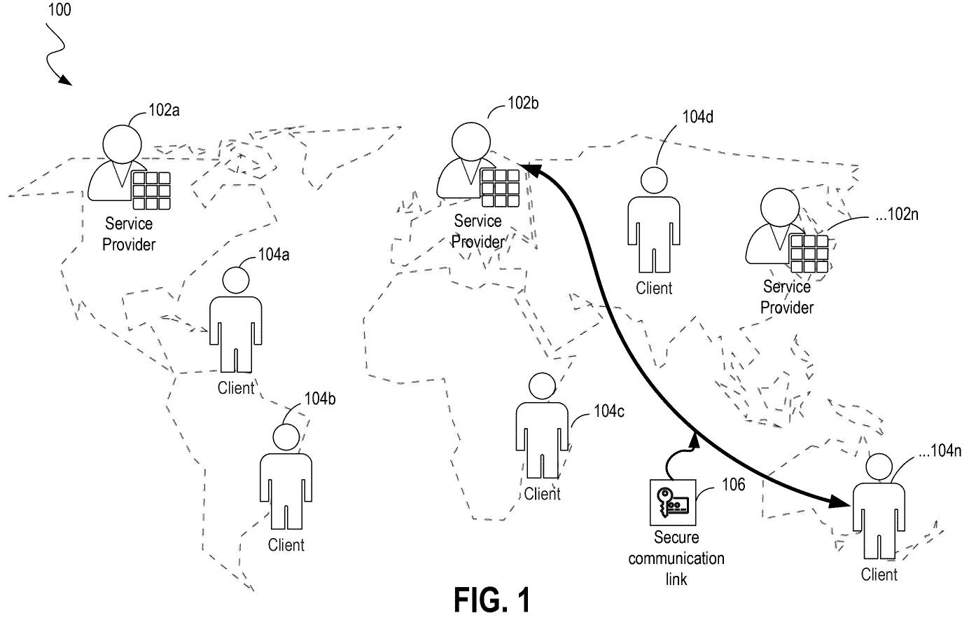

FIG. 1 illustrates a world map 100 with a client in one jurisdiction consulting a service provider in a different jurisdiction of the world, in accordance with at least one embodiment. In many jurisdictions, particularly in rural, remote, or underserved areas, certain cadres of specialist services providers may not be available. In at least one embodiment, AI based online consultations can help clients to understand their needs and then identify service providers, registered with the consultation services database, which can help resolve the challenges faced by clients. In at least one embodiment, service providers may not be residing in the city where the clients are, rather they might be in any part of the world where they can connect to the Internet.

At least one example provides a ubiquitous platform, where one or more service providers 102a, . . . , 102n, residing in different geographical jurisdictions, may be consulted online by one or more clients 104a, . . . , 104n from any place where they could connect to the Internet. Consequently, in scenarios where a subject might need consultation with a competent and certified professional (e.g., a healthcare profession) residing in a distant jurisdiction, the subject can use the AI based ubiquitous consultation platform to seek help and guidance on their issue (e.g., health state). As a result, accurate and timely diagnosis is made without delay in a cost-effective manner, as the subject may not need to travel the country of service providers (e.g., healthcare providers). Typical delays can be caused by visa approvals and travel delays. Such delays can be reduced. In at least one embodiment, a client 104n residing in Australia can use AI based ubiquitous consultation services (e.g., through audio or video) to seek help from a service provider 102b residing in Europe over a secure encrypted communication link 106.

In at least one embodiment, a database of one or more healthcare providers is created as service providers. Healthcare providers, residing in any geographical jurisdiction, may be invited to provide personal and professional credentials. The credentials may include name, age, gender, location, nationality, field of specialty, degree, license, years of experience, feedback from previous subjects, malpractice history, lawsuits by subjects, prior disciplinary actions, prior judgements, and insurance history of denial of medical claims. One or more healthcare providers may be verified by validating a set of professional credentials from online databases, maintained by relevant regulatory bodies or authorities. One or more verified practitioners may be registered and stored in the database.

In at least one embodiment, a database of one or more automobile mechanics is created. Automobile mechanics, residing in any country, may be invited to provide personal and professional credentials. The credentials may input name, age, gender, location, nationality, field of specialty, mechanical degree, years of experience, feedback from previous clients, etc. For example, field of specialty for automobile mechanics may include specialties like diesel engine, gasoline engine, electric cars, batteries, suspension, transmission, body work, electrical systems, sensors, tuning, wheel alignment and balancing, cooling system, air conditioning, exhaust, gearbox, steering etc. One or more automobile mechanics may be verified by validating the set of professional credentials from online databases maintained by relevant regulatory bodies or authorities. The one or more verified mechanics may be registered and stored in a database.

In at least one embodiment, one or more databases of service providers relating to plumbers, heating ventilation, and air conditioning (HVAC) technicians, civil workers, grocery shoppers, pharmacists, sewerage workers, gardeners, welders, metal workers, wood workers, cobblers, hair stylist, beauticians, sanitary workers, tourism agents, travel agents, interior designers, computer software engineers, computer hardware engineers, etc. from all over the world may be created.

In at least one embodiment, a computer-implemented method is provided that receives an electronic input from a client, wherein the electronic input may include a problem description by the client. In at least one embodiment, the problem description may involve concerns about the health state of the client. The condition may be described in a natural language text or voice format. In at least one embodiment, electronic health records (EHRs) of the client are obtained from one or more EHR providers. In at least one embodiment, a set of attributes, associated with the client from one or more EHR providers is accessed, wherein the set of attributes may include one or more attributes of the client, such as personal information, demographics, ethnicity, gender, language, health profiles data, insurance policy, and geographic location.

In at least one embodiment, the problem description may be malfunctioning of an automobile described in a natural language text or voice format. A set of attributes associated with the automobile may be obtained including the model, mileage, a log of previous maintenance records, etc.

In at least one embodiment, one or more machine-learning (ML) models are trained to extract the problem description from the natural language text or voice input. In at least one embodiment, based on the problem description and the set of attributes, a subset of one or more service providers is identified from the database of service providers. In at least one embodiment, the subset of one or more service providers may include plumbing specialists. In at least one embodiment, a plumber may be selected based on the problem described by a client and the client's personal preferences. In at least one embodiment, an appointment of the client with the plumber for a videoconferencing session may be scheduled.

In at least one embodiment, the problem statement can be seeking assistance from a healthcare provider about the health state of a subject. In at least one embodiment, based on the condition and the set of attributes, a subset of the one or more healthcare providers is identified from the database who are verified and certified from a relevant healthcare regulatory body. In at least one embodiment, a provider may be selected from the subset of providers based on the subject preferences, subject attributes, and subject health condition. In at least one embodiment, an appointment over a video conferencing session is scheduled between the client and the provider.

In at least one embodiment, there is a provision for the client and the service provider to share data in real time during the video conference session using document sharing services of the video conference application. In at least one embodiment, an audio conversation during the video conferencing session may be transcribed, in real-time, by leveraging one or more speech recognition models. In at least one embodiment, by applying one or more machine-learning models, the problem of a subject is determined from the transcription of the audio. In at least one embodiment, the problem may be related to a health condition of a client. In at least one embodiment, one or more health attributes, defining the digital health state of a subject, may be accessed from the EHR to contextualize the audio conversation using the health history of a subject. In at least one embodiment, the management interventions related to the health condition of a subject may also be transcribed based on the one or more attributes during the real-time video conferencing session. In at least one embodiment, the electronic input is processed by one or more NLP models, to extract an intent of the electronic input.

In at least one embodiment, a computer-implemented method is tangibly embodied in a non-transitory machine-readable storage medium, including instructions configured to cause one or more data processors to perform part or all of one or more methods or processes disclosed herein. In at least one embodiment, a system is provided that includes one or more means to perform part or all of one or more methods or processes disclosed herein.

In at least one embodiment, a secure communication link 106 is used to ensure security and privacy of sensitive personal data of subjects, shared during ubiquitous consultations with service providers, by providing an end-to-end secure encrypted link. In case of health care service providers and patients as clients or subjects, strict data security requirements of general data protection regulation (GDPR) in Europe and the health insurance portability and accountability act (HIPAA) in the United States must be complied with when handling subjects' data especially related to healthcare data. Compliance with GDPR, HIPPA, and other such compliant regulations can gain confidence in the consultation services as clients will feel assured that their personal data will not be misused. In at least one embodiment, adequate data security measures are put in place that will make it very difficult for malicious hackers to compromise the consultation services by launching cybersecurity attacks and the security firewalls. In at least one embodiment, the subjects' data is encrypted, using the prescribed encryption standard like AES 128, 192, or 256, at rest and in transit to provide confidentiality of data.

The following examples are described with reference to a health care provider (e.g., nurse, physician assistant, doctor, or surgeon) as a service provider and a patient as a client or subject. The methods and associated hardware for ubiquitous consultations are applicable to other service providers (e.g., electrician, plumber, motor vehicle mechanic, architect, landscaper) and clients.

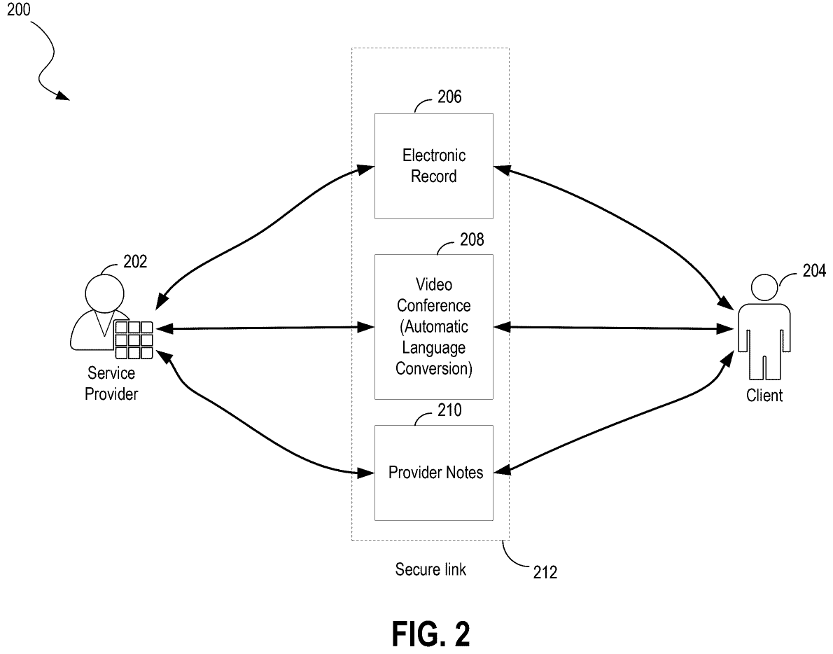

FIG. 2 illustrates an AI based ubiquitous consultation session 200 of consultation between a service provider 202 and a client 204, in accordance with at least one embodiment. In at least one embodiment, service provider 202 is a healthcare professional who analyzes the healthcare state of client 204. In at least one embodiment, the digital health state of client 204 is stored in an electronic health record (EHR) system 206 of service provider 202. In at least one embodiment, EHR system 206 may include one or more electronic health records of client 204, which are aggregated and sent to service provider 202. In at least one embodiment, EHR system 206 may also have additional healthcare data of client 204, which is uploaded by client 204 from a local computing device. In at least one embodiment, aggregated electronic healthcare records of client 204 may be shared with service provider 202 when requested.

In at least one embodiment, AI based ubiquitous consultation session 200 may establish a video conference 208 session between the service provider 202 and client 204 by scheduling an appointment with service provider 202 from the available slots of service provider 202. In at least one embodiment, conversation during the video conferencing consultation is transcribed using one or more AI based speech processing models. Examples of such AI based speech processing models include hidden Markov models (HMMs), gaussian mixture models (GMMs), deep neural networks (DNNs), convolutional neural networks (CNNs), recurrent neural networks (RNNs), connectionist temporal classification (CTC) etc.

HMMs model speech as a sequence of states, with each state corresponding to a phoneme or sub-phonetic unit. In at least one embodiment, HMMs are trained in a supervised mode to learn the probabilities of transitioning between states by outputting acoustic features. In at least one embodiment, DNNs are used to directly map acoustic features to phoneme probabilities or word sequences. In at least one embodiment, CNNs are used to extract hierarchical features from spectrogram representations of speech signals. In at least one embodiment, RNNs, such as Long Short-Term Memory (LSTM) networks, are used for sequence data including speech. In at least one embodiment, RNNs are used to capture temporal dependencies within speech signals and are commonly used in acoustic modeling and natural language modeling systems.

In at least one embodiment, conversation during video conference 208 is transcribed by leveraging one or more machine learning models configured for speech recognition. For example, DNNs may be used to map acoustic features extracted from audio signals to phoneme probabilities or directly to word sequences. RNNs, like LSTM and gated recurrent unit (GRU) may be used to model temporal dependencies in speech signals and language context. Sequence-to-sequence models, based on encoder-decoder architectures with attention mechanisms may directly map variable-length input speech signals to variable-length output text transcripts.

Service provider 202 and client 204 may speak different languages. In at least one embodiment, AI based ubiquitous consultation session 200 may provide real-time automatic language translation services between different languages to allow service provider 202 and client 204 to better understand each other. In at least one embodiment, speech is transcribed into text by leveraging speech recognition models RNNs or transformers, which analyze the audio input and convert it into corresponding text. In at least one embodiment, natural language processing (NLP) techniques are used to understand the semantics of the transcribed text and extract relevant content, which may include language identification, part-of-speech tagging, parsing, and entity recognition. In at least one embodiment, machine translation models, particularly statistical machine translation or neural machine translation (NMT) models are used to translate the transcribed text into a target language. In at least one embodiment, the translated text is converted into audio in real-time using text-to-speech synthesis (TTS) systems of the relevant language to generate natural sounding human speech by concatenating pre-recorded speech segments or by using neural network based models to generate speech waveform directly from text.

In at least one embodiment, AI based ubiquitous consultation session 200 uses one or more machine learning models for language translations. Examples of such machine learning models include rule-based translation (RBT), statistical machine translation (SMT), neural machine translation (NMT), NLP, and incremental translation. RBT uses predefined linguistic rules and dictionaries to perform language translation and may rely on grammatical and syntactical rules of both languages involved. SMT may use statistical models to translate speech. SMT may analyze large corpora of texts from both languages to learn patterns and probabilities of occurrence of word sequences. SMT can handle complex language structures and idiomatic expressions. SMT translation may be word by word or phrase by phrase. NMT may use deep learning techniques, specifically deep neural networks, to translate speech. NMT may consider the whole sentence context, and may produce fluent and accurate translations, especially for languages with complex grammar. NLP techniques may be employed to understand the meaning of the transcribed text, considering context, grammar, and semantics. In at least one embodiment, language translation is performed by leveraging one or more AI tools, for example, Google® translate, Microsoft® translator, or Worldly® etc.

Conversation during the video call may relate to healthcare. At the end of AI based ubiquitous consultation session 200, the apparatus and method discussed herein use one or more machine-learning (ML) models to generate provider notes 210. In at least one embodiment, ML models extract medical terminology and intents from transcription of the conversation during video conference 208. The one or more ML models may include NLP models, LLMs, and specialized healthcare NLP models. In at least one embodiment, the speech is tokenized, and features are extracted using feature representation techniques including bag-of-words, TF-IDF (Term Frequency-Inverse Document Frequency), word embeddings (e.g., Word2Vec, GloVe), or contextual embeddings (e.g., BERT, ELMO).

One or more pattern classification methods may be used for intent classification. For example, logistic regression, support vector machines (SVM), decision trees, random forests, gradient boosting, or neural network architectures such as feedforward networks, RNNs, CNNs, or transformer models etc. In at least one embodiment, transfer learning with large pre-trained language models is fine-tuned on healthcare-specific data for identification of speakers' intent in a healthcare service, for example, bidirectional encoder representations from transformer models like BERT or generative pre-trained transformer GPT.

In at least one embodiment, provider notes 210 may also include information extracted from electronic health record of client 204 by leveraging one or NLP models. In at least one embodiment, clinical notes are generated from electronic health records by performing preprocessing, named Entity Recognition (NER), concept mapping, Semantic Role Labeling (SRL), template filling, and language generation. Preprocessing may include removal of irrelevant information from subjects' records (such as headers, footers, or other non-clinical content), tokenization, and sentence segmentation. Pretrained NER models may identify and extract entities such as subject names, medical conditions, medical prescriptions, procedures, and dates from medical records. Concept mapping may map extracted entities to standardized medical terminologies such as SNOMED CT or RxNorm. In at least one embodiment, predefined templates can be used by a healthcare provider to enter sections such as subject's history, physical examination, assessment, plan, etc. In at least one embodiment, generative AI based models are used to generate provider notes 210 that describe the history and digital health state of a client 204 in a target language and store them in EHR system 206. In at least one embodiment, provider notes 210 can be reviewed by a different healthcare provider 102a, when client 204 wants to consult healthcare provider 102a for an online consultation.

In at least one embodiment, the proceedings of the online consultation session 200 may be encrypted at rest and at transit on secure link 212. In at least one embodiment, encryption of data at rest may be implemented using AWS® RDS and encryption of data during transit can be implemented using secure sockets layer SSL protocol.

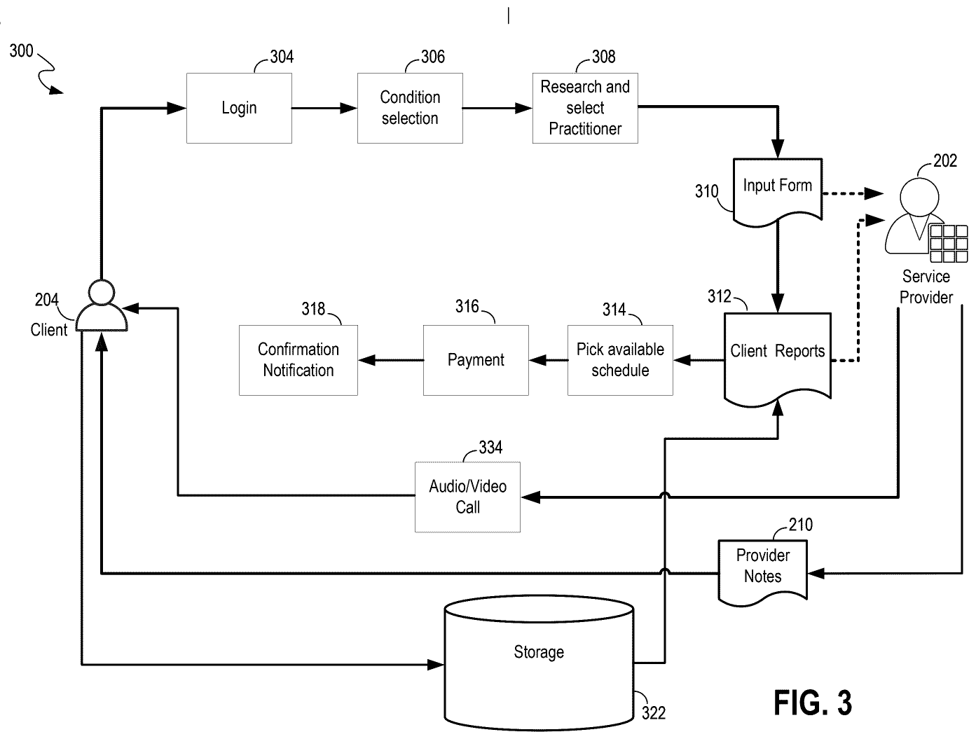

FIG. 3 illustrates a computerized method and associated hardware (herein of online consultation system 300) client initiating consultation with a service provider, in accordance with at least one embodiment. Online consultation system 300 may include a login interface 304, a list of health conditions 306, a list of practitioners 308, an input form 310, a client report 312, a schedule 314, a payment processing unit 316, notification 318, audio and/or video call 334, provider notes 210, and storage database 322. Various blocks of online consultation system 300 may be implemented in software, hardware, or a combination of both. For instance, online consultation system 300 may be a smart phone, laptop, tablet, or desktop.

In at least one embodiment, client 204 initiates interaction with online consultation system 300 by triggering login interface 304. In at least one embodiment, login interface 304 is a software interface that may use facial recognition, text-based user-id and password, fingerprint, or any other login method to access online consultation system 300. In at least one embodiment, login interface 304 may be displayed on a screen for client 204 to interact. Login interface 304 may be presented in written or verbal language understandable by client 204. In at least one embodiment, login interface 304 may apply an authentication process to verify client 204.

After client 204 successfully logs into online consultation system 300, an AI digital assistant queries client 204 to infer likely health condition(s) of client 204. In at least one embodiment, the AI digital assistant generates a list of health conditions 306 that client 204 may be suffering from and presents it to client 302. Client 204 may select one or more conditions from list of health conditions 306 that closely match his health state. In at least one embodiment, client 204 may also describe their health condition in a text form and/or by speaking to online consultation system 300. In at least one embodiment, online consultation system 300 presents a visual interface that includes logic interface 304 (which may disappear after successful logic), a region where list of health conditions 306 are presented, a region where list of practitioners 308, input form 310, client report 312, and other information herein is presented.

In at least example, one or more NLP techniques are used to extract and classify the intent of client 204. The classification of intent may identify the type of intent, for example, whether the intent corresponds to vitals and symptoms, diagnosis, treatment plans, health state information, possible preventive measures, or scheduling an online consultation session with a suitable service provider 202 based on health conditions 306 of client 204. In at least one embodiment, list of practitioners 308 including one or more service providers is presented, from which client 204 may select a suitable service provider 202. In at least one embodiment, the selection of service providers 202 can also be done by the AI digital assistant by factoring in attributes or merits associated with a service provider and client preferences. Examples of attributes or merits associated with a service provider include specialty, degree, license, experience, feedback from previous clients, malpractice lawsuits, prior disciplinary actions, prior lawsuit judgements, and insurance coverage etc. Examples of client preferences include ethnicity, insurance coverage, native language, and/or gender preferences etc.

In at least one embodiment, client 204 can also complete input form 310 to enable service provider 202 to better understand the health condition of client 204. Input form 310 may include queries related to smoking, alcohol consumption, dietary restrictions, allergies to medicine or food, etc., or any other healthcare conditions experienced in the past, any questions or concerns, and physical activity of client 204. In at least one embodiment, physical activity or gym data may be obtained from a client's smart phone sensors, smart watches, or gym equipment usage data, such as Peloton®.

In at least one embodiment, online consultation system 300 can, using the data entered in input form 310 and the information provided in the natural language, generate structured electronic health record of client 204 and store in an EHR system at storage database 322. Online consultation system 300 may use one or more EHR providers, for example, EPIC® or CERNER® to create EHR system at storage database 322. The electronic health record of client 204 may be pulled from the EHR system at storage database 322, edited and updated by service provider 202 and/or client 204, and then saved in EHR system at storage database 322. One or more electronic health records of client 302 can be aggregated to create comprehensive client reports 312 and then share with service provider 202.

In at least one embodiment, online consultation system 300 presents available schedule 314 of service provider 202 to client 204 to schedule an online consultation appointment. In at least one embodiment, available schedule 314 is presented in time zones of client 204 and service provide 204. After client 204 schedules an appointment time from available schedule 314, online consultation system 300 presents a portal for payment processing unit 316. Payments can be made using any suitable means including credit card, bank account, cryptocurrency, etc.

After payment is made, online consultation system 300 sends a notification 318 confirming the scheduled appointment to client 204 and service provider 202. In at least one embodiment, notification 318 may be an email or a text message and may include a link to the online audio/video consultation. In at least one embodiment, the consultation link can be a ZOOM® link, or Microsoft Teams link, or Google Meet link, or some other suitable audio and/or video application. In at least one embodiment, at the scheduled time, online consultation system 300 may initiate audio/video call 334 between service provider 202 and client 204, and both can join the consultation by clicking on the consultation link.

In at least one embodiment, the conversation during audio and/or video call 334 relates to healthcare and wellness advice between client 204 and service provider 202. In at least one embodiment, provider notes 210 are generated by a suitable generative AI or NLP model by transcribing the conversation in audio/video call 334. In at least one embodiment, the one or more machine learning models that generate lists of practitioners and provider notes 210 may be running on premises locally on central processing units (CPUs), one or more graphical processing units (GPUs), one or more cloud-based CPUs, one or more cloud-based GPUs, or a combination thereof.

During audio and/or video call 334 between client 204 and service provider 202, suitable generative AI or NLP model generates provider notes 332 that may contain information, such as medical history of a subject, list of diagnoses with reasonings, treatment plans with references and citations, medicine instructions, and follow-up actions. In at least one embodiment, provider notes 332 for client 204 may include information such as: “Subject X presents with hypertension, BP 150/95 mmHg. Subject has a family history of cardiovascular disease. Subject is prescribed antihypertensive medication X, 10 mg once daily. Subject is advised to change his lifestyle by reducing sodium intake and doing aerobic exercise for 30 minutes daily. Subject is advised to come for a follow-up visit after 4 weeks to assess the outcome of the recommended interventions and change the treatment plan if need be.”

In at least one embodiment, online consultation system 300 generates a summary of provider notes 332 for client 204. The summary may include main information elements, which define the digital health state of client 204, a management plan including a treatment plan, and follow up visits. In at least one embodiment, online consultation system 300 sends a copy of the summarized note to client 204 by email or cell phone text and stored in storage of EHR system at storage database 322.

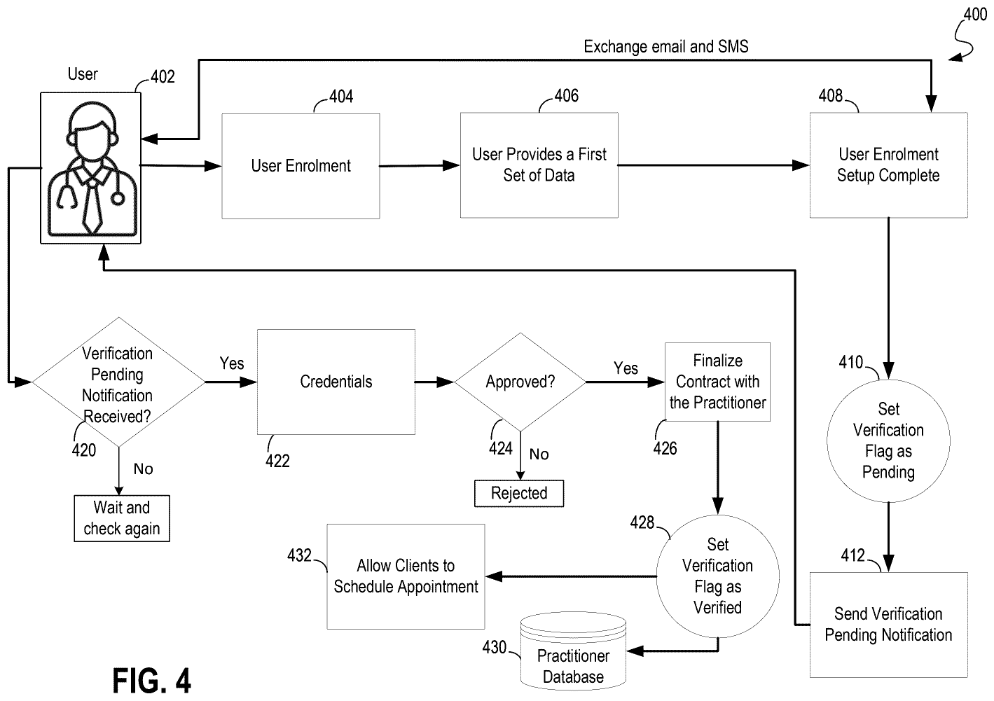

FIG. 4 illustrates a secure method and associated hardware of registration and approval of one or more service providers, in accordance with at least one embodiment. In at least one embodiment, secure method and associated hardware of registration and approval is a registration workflow 400 where service provider 204 is registered and approved for online consultation system 300. Service providers worldwide may be searched on the internet to obtain contact information. Service providers, in different domains, may be contacted and invited through emails or text messages to register on the online consultation system 300. Various blocks of registration workflow 400 may be performed by hardware, software, or a combination of these.

In at least one embodiment, after receiving the invitation, a service provider 402 enrolls as a potential service provider by completing a user enrollment 404. Information for user enrollment 404 includes username, password, email, and phone number, etc. In at least one embodiment, service provider 402 is asked to enter a first set of data 406 which may comprise name, address, date of birth, demographic data, field of specialization, state license, etc. After entering first set of data 406 and user verification, user enrollment setup is deemed complete as indicated by block 408. At block 408, an email and/or cell phone message with an email verification link is sent to the email address and mobile phone of service provider 402. Service provider 402 can click on the email verification link to complete the enrolment setup. In at least one embodiment, a verification flag status is set to “pending” at block 410. In at least one embodiment, a verification pending notification 412 is sent to service provider 402 to inform service provider 402 about the pending status of verification. In at least one embodiment, service provider 402 checks the verification pending notification at block 420. In at least one embodiment, if verification pending notification 412 is received, service provider 402 may be asked to enter a second set of information elements at block 422. In at least one embodiment, if verification pending notification 412 is not received, the system waits and checks for notification again. If the notification is not received after waiting for a predetermined or programmable time, service provider 402 is informed.

In at least one embodiment, the second set of data includes credentials that are provided online by service provider 402. In at least one embodiment, credentials may include service provider specialty, educational background, degree, graduation date, license, experience, feedback from previous subjects, malpractices lawsuits, prior disciplinary actions, prior judgements, insurance coverage, etc. In at least one embodiment, credentials are analyzed for the authenticity of the credentials by searching a data bank such as the National Practitioner Data Bank (NPDB) of a country.

In at least one embodiment, in the case where service provider 402 is a medical practitioner, educational background including degree, graduation date, licensing, medical school attended, residency training, and any fellowships or additional training may be confirmed from online databases of healthcare licensing boards or colleges, regulatory authorities, or professional associations of different countries. Examples of regulatory authorities and licensing blocks include Antigua & Barbuda Medical Council in Antigua & Barbuda, Punjab Medical Council in Punjab India, Pakistan Medical and Dental Council in Pakistan, etc., where specialized trainings and certifications can be confirmed from relevant specialty boards or professional organizations. Examples of specialty boards or professional organizations include European Society of Cardiology, Chinese Cardiovascular Association, American Orthopedic Association, Australian Knee Society, Society of Surgical Oncology, Indian Society of Oncology, etc.

In at least one embodiment, if the credentials from block 422 are approved, a contract 426 is finalized with service provider 402. Otherwise, service provider 402 is rejected and may be asked to reapply after their credentials are verifiable. Upon completing contract 426, the verification pending flag may be set as “verified” at block 428. In at least one embodiment, service provider 402 may be registered and added to a service provider database 430 after the verification pending flag is set to “verified” at block 428. The process then proceeds to block 432. At block 432, subjects such as client 204, may be allowed to schedule appointments with the verified service providers who are registered in service provider database 430.

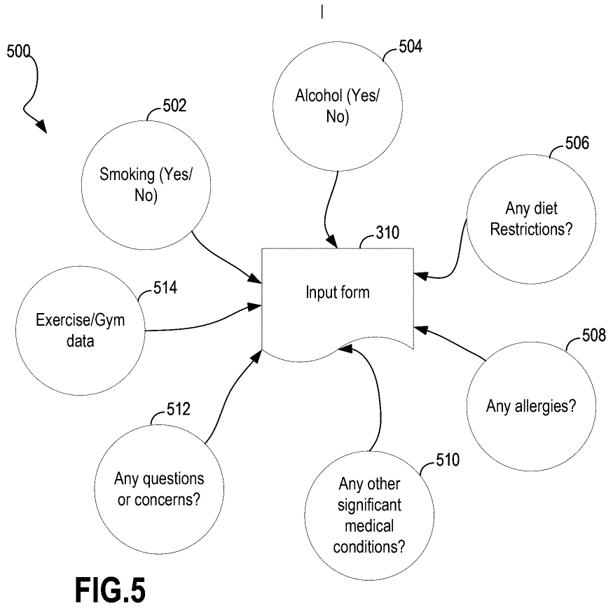

FIG. 5 illustrates input form 310 to be completed by client 204, in accordance with at least one embodiment. Input form 310 is used for collecting information from client 204 to better understand their online consultation requirements. In at least one embodiment, where an online health consultation is requested, the input data may include queries about smoking history 502, alcohol consumption 504, dietary restrictions 506, allergies to medicine or food 508, etc., other significant medical conditions 510, questions or concerns 512, and exercise or gym data 514, which can be obtained from client's smart phone sensors, smart watches, or gym equipment, such as a Peloton® data provider.

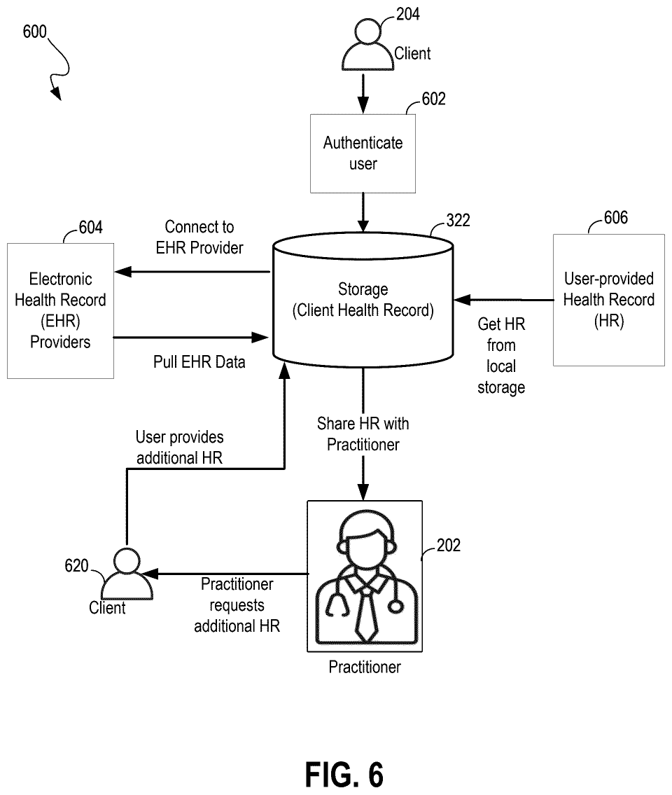

FIG. 6 illustrates a method and associated hardware (herein system 600) of preparing consolidated medical record of a subject, in accordance with at least one embodiment. Various blocks described herein can be performed by software, hardware, or combination thereof. System 600 is used to prepare electronic healthcare records of client 204, which are stored in the storage database 322 of online consultation system. System 600 may provide authentication at block 602 to enable client 204 to securely connect to one or more EHR providers 604. In at least one embodiment, EHR providers 604 may provide one or more verified electronic health records of client 204 stored in storage database 322 of online consultation system 300. In at least one embodiment, client 204 may upload some additional informational elements of their electronic healthcare record, stored on storage 606 of their local computing device, in response to a request from service provider 202. Service provider 604 may merely trust client 204 because it is a challenge to verify that the uploaded information elements of electronic health record belong to client 204. The aggregated electronic healthcare records are then shared securely with service provider 202 who can pull the relevant EHR data that is stored in storage database 322.

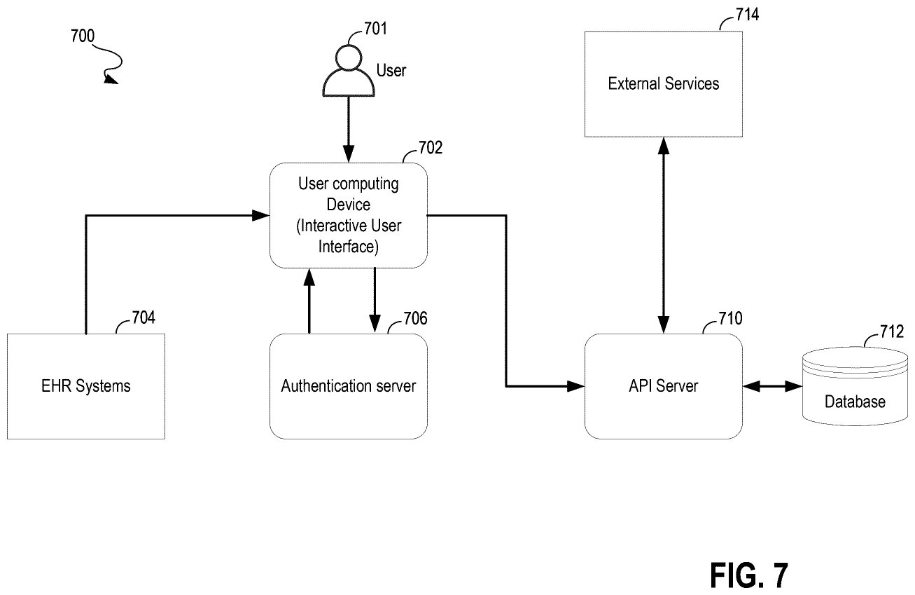

FIG. 7 illustrates a computer architecture 700 for secure electronic sessions for consultation between client and service providers, in accordance with at least one embodiment. Computer architecture 700 comprises computing device 702, EHR system 704, authentication server 706, application programming interface (API) server 710, and database 712. A user 701 may use computing device 702 to securely login into online consultation system 300 by entering access credentials at a user interface screen on computing device 702. In at least one embodiment, the user interface can be implemented in React, Angular, or Flutter. In at least one embodiment, the user is authenticated with the help of authentication server 706 which may be running compute instances of Okta servers or Lightweight Directory Access Protocol (LDAP) Servers. User authentication servers manage users' access credentials, validate authentication requests, and grant access when the identity of user 701 is verified. After authentication, user 701 may pull their EHR data from EHR system 704. In at least one embodiment, backend functionality is provided by API server 710, like Amazon API Gateway, RESTful API Servers, or GraphQL API Servers, etc. In at least one embodiment, API server 710 can be responsible for handling requests from users' applications, processing requests, generating responses, and sending them back to users. In at least one embodiment, API server 710 may serve as the bridge between a front user interface at computing device 702, database 712, and one or more external services 714. In at least one embodiment, API server 710 connects the user to the one or more external services 714, which may include secure electronic payment services like STRIPE®, scheduling services like BOOKAFY®, audio/video sessions like ZOOM®, and email services like SendGrid®. In at least one embodiment, API Server 710 provides access to database 712.

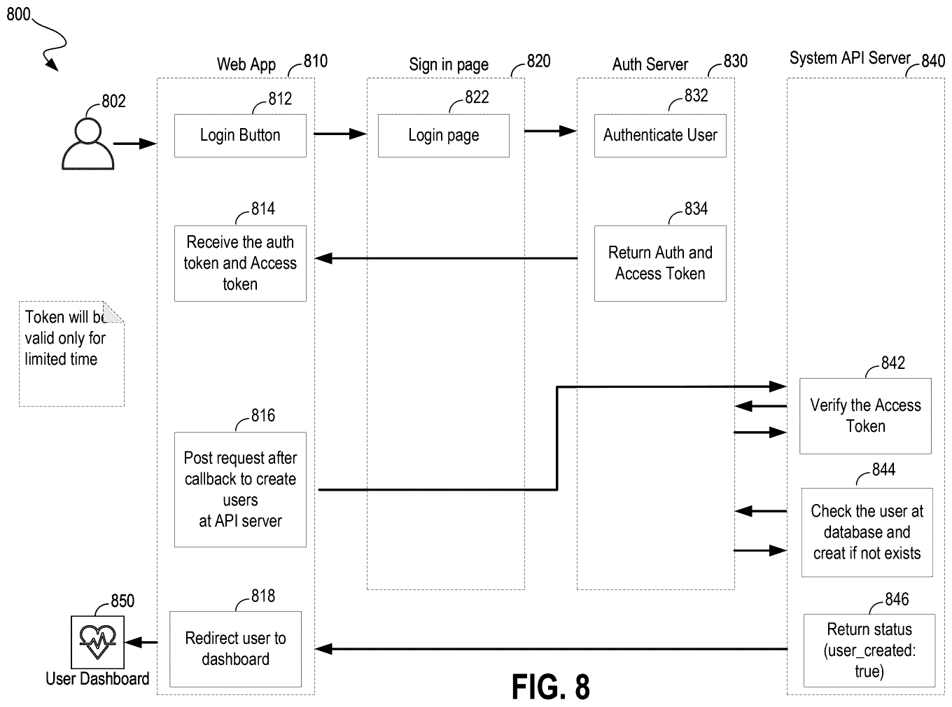

FIG. 8 illustrates a web application process and associated hardware (herein workflow process 800) to sign into a system application programming interface (API) server, in accordance with at least one embodiment. In at least one embodiment, workflow process 800 is used for a user 802 (e.g., client 204 or service provider 202) to access an API server 840. In at least one embodiment, user 802 opens web application 810 at a local device and clicks a login button 812. In response, a login page 822 can be shown. In at least one embodiment, user 802 enters personal access credentials at login page 822, such as username and password. In at least one embodiment, personal access credentials are sent to an authentication server 830 that is responsible for verifying the identity of users and devices who are trying to access resources of a computer network in a secure manner. Authentication server 830 may provide services like user credential verification, authentication process, security protocols, logging and auditing, and integration with other systems. Some authentication service applications include Microsoft® Active Directory, Cisco® Identity Services Engine (ISE), FreeRADIUS®, or Amazon® Cognito, etc. In at least one embodiment, authentication server 830 authenticates user 802 at box 832 and returns an authentication token and access token at box 834 to web application 810 that is received by web application 810 at box 814. In at least one embodiment, at box 814, the authentication token and access token are stored at a local storage that is managed securely, and a user session may be started. At box 816, web application 810 can post a request to create accounts for legitimate user 802 at API server 840. In at least one embodiment, at box 842, API server 840 verifies the access token by using authentication server 830. In at least one embodiment, at box 844, API server 840 checks with authentication server 830 whether an account for legitimate user 802 exists, if not then it creates an account for user 820. In at least one embodiment, at box 846, API server 840 sends a user's account status—created or existing—to web page application 810, which is received at box 818. In at least one embodiment, at box 818, user 802 is redirected to a user dashboard 850.

In at least one embodiment, user dashboard 850 is a personalized interface or control panel within user web app 810 that may provide user 802 access to relevant information, tools, and settings. User dashboard 850 may provide services like account information, settings, activity feed, content management, communication tools, billing and payments, help and support, integration with third-party services, and security features etc. In at least one embodiment, user 802 uses dashboard 850 to view and manage account details and credentials: username, email address, profile picture, and contact information. In at least one embodiment, dashboard 850 provides tools to user 802 to manage content, such as creating, editing, or deleting posts, documents, images, or other media. In at least one embodiment, dashboard 850 provides access to messaging systems, chat features, video conferencing, or forums where user 802 may interact with other users or with customer support. In at least one embodiment, using dashboard 850, user 802 can view billing history, manage payment methods, or access invoices, etc. In at least one embodiment, dashboard 850 provides settings related to account security, such as password management, two-factor authentication, or security alerts, etc.

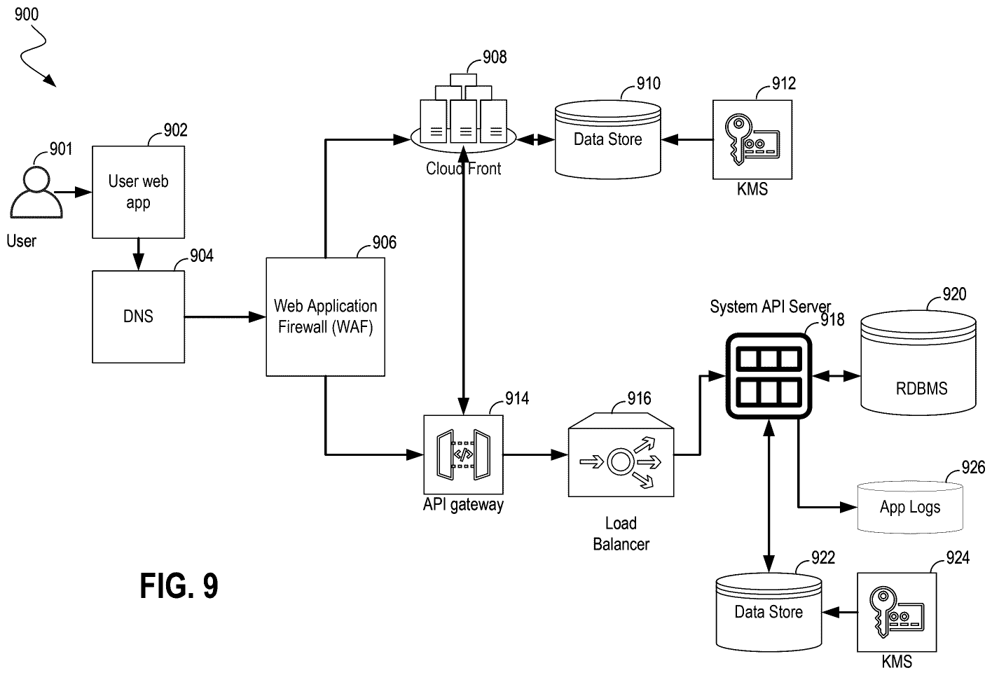

FIG. 9 illustrates a system 900 and associated workflow that enables a user 901 (e.g., client 204 or service provider 202) to use resources of online consultation services platform. In at least one embodiment, user 901 starts a user web application 902 which can be a ubiquitous online consultation service discussed herein. In at least one embodiment, a domain name system DNS server 904 redirects user web application 902 to a cloud front 908. DNS server 904 may be Amazon® AWS Route 53, Google® cloud DNS, Cloudflare, or Microsoft® Azure DNS, etc. In at least one embodiment, a web application firewall (WAF) 906 sits between cloud front 908 and DNS server 904 to implement application security like demographic restrictions to prevent attacks exploiting known vulnerabilities, such as SQL injection, cross-site scripting (XSS), file inclusion, etc. In at least one embodiment, cloud front 908 connects to a data store 910 and may send the response back to user 901. In at least one embodiment, data store 910 may be Google® cloud storage, Microsoft® Azure blob storage, IBM® cloud object storage, Alibaba® cloud object storage service (OSS), or Oracle® cloud infrastructure object storage, etc.

In at least one embodiment, data store 910 may be encrypted with a key management service (KMS) 912. In at least one embodiment, KMS 912 may be AWS Key Management Service, Google® Cloud Key Management Service, Microsoft® Azure Key Vault, Alibaba® Cloud Key Management Service, or Oracle® Cloud Infrastructure Key Management, etc. Web applications may be stored at a data store 910 and may be served to user 901 via a cloud front 908.

API gateway 914 receives a request from user 901 and forwards it to an API server 918, which may be a server providing online consultation services. In at least one example API gateway 914 may be Amazon® API gateway, Google® cloud endpoints, Microsoft® Azure API management, or IBM® API connect, etc. In at least one embodiment, API gateway 914 routes incoming API requests from user 901 to the appropriate backend services hosted at API server 918. In at least one embodiment, API gateway 914 configures API security. In at least one embodiment, API Gateway 914 transforms incoming requests and outgoing responses to match the format expected by backend services hosted at API server 918 or user web app 902. In at least one embodiment, API Gateway 914 provides mechanisms to secure APIs and control access to backend resources at API server 918. In at least one embodiment, API Gateway 914 integrates with various backend services like accessing a data store 922 of a relational database management system RDBMS 920, and associated application logs 926.

In at least one embodiment, a load balancer 916 like AWS Load balancer is used to route traffic between multiple API servers running on API server 918. In at least one embodiment, NGINX server running on API server 918 distributes incoming traffic across multiple backend servers to ensure high availability and resource utilization. In at least one embodiment, API server 918 runs a web server like NGINX and an application server like PUMA. In at least one embodiment, API server 918 executes on a virtual machine service such as Amazon® machine image AMI, Google® cloud platform GCP, or Oracle® cloud infrastructure OCI, etc.

In at least one embodiment, data storage 922, coupled to system API server 918, has application assets like images, pdfs, and other resources relevant to user 901. Data storage 922 may be Amazon S3 Bucket. In at least one embodiment, the relational database management system RDBMS 920 provides data encryption at rest and during transit. At rest, a disk may be encrypted, and in transit SSL encryption may be applied. In at least one embodiment, the RDBMS 920 can be MySQL, PostgreSQL, Oracle® database, or Microsoft® SQL server, etc.

In at least one embodiment, application logs are stored at App Logs 926 which can be AWS Cloud Watch, Google® cloud monitoring, or Microsoft® Azure monitor, etc. Application logs may include records that are generated by software applications, servers, or systems that capture events, actions, errors, and other relevant information during operations like event logs, error logs, access logs, or debug logs, etc. Application logs may be used for troubleshooting, debugging, monitoring, and analyzing the behavior of applications and systems.

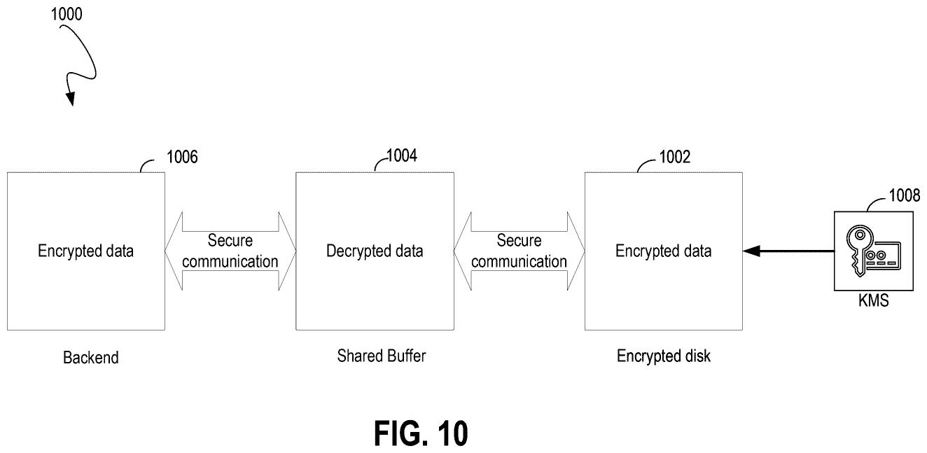

FIG. 10 illustrates a method 1000 of data encryption and associated hardware for online consultation services application, in accordance with at least one embodiment. In at least one embodiment, an encrypted disk 1006 contains encrypted data at rest. In at least one embodiment, data is written to encrypted disk 1006 by encrypting the data using an encryption algorithm with a private key, and legitimate users can decrypt the data as they have access to the private key. In at least one embodiment, the private key used for encryption of, and decryption of data are provided by a Key Management Service (KMS) 1008. In at least one embodiment, KMS 1008 may be AWS Key Management Service, Google® Cloud Key Management Service, Microsoft® Azure Key Vault, Alibaba® Cloud Key Management Service, or Oracle® Cloud Infrastructure Key Management, etc.

In at least one embodiment, at backend, data is encrypted using Advanced Encryption Standard (AES), Java with Spring Boot, Python with Django or Flask, Node.js with Express, etc. In at least one embodiment, a requested piece of encrypted data is decrypted using decryption key provided by KMS 1008, and the decrypted data is stored in a shared buffer 1004.

Encrypting data in transit may be used for ensuring data security as data travels between different networks of different geographic jurisdictions during online consultations between a user and a server or between servers. In at least one embodiment, encryption of data in transit is implemented using secure sockets layer (SSL), transport layer security (TLS), secure shell (SSH), Internet Protocol Security (IPsec), Virtual Private Network (VPN), hypertext transfer protocol secure (HTTPS), etc. SSL and TLS are cryptographic protocols that provide secure communication over a network and may encrypt data exchanged between a client (such as a web browser) and a server (such as a website) to ensure confidentiality and integrity. IPsec is a suite of protocols that can be used to secure Internet Protocol (IP) communications by authenticating and encrypting IP packets of a communication session. SSH is a cryptographic network protocol that may be used for secure remote login, remote command execution, and other secure network services between two networked devices. SSH may encrypt data transmitted over the network, preventing eavesdropping and tampering. IPsec may be used for securing virtual private networks (VPNs) and other network connections. A VPN may create a secure, encrypted connection over a less secure network, such as the internet. VPN may allow users to access private networks and resources securely while encrypting data in transit between the user's device and a VPN server. HTTPS is an extension of HTTP that uses SSL/TLS encryption to secure data transmitted between a web browser and a web server. HTTPS may be used for secure communication on the world wide web, such as online banking, e-commerce, and sensitive data transfers.

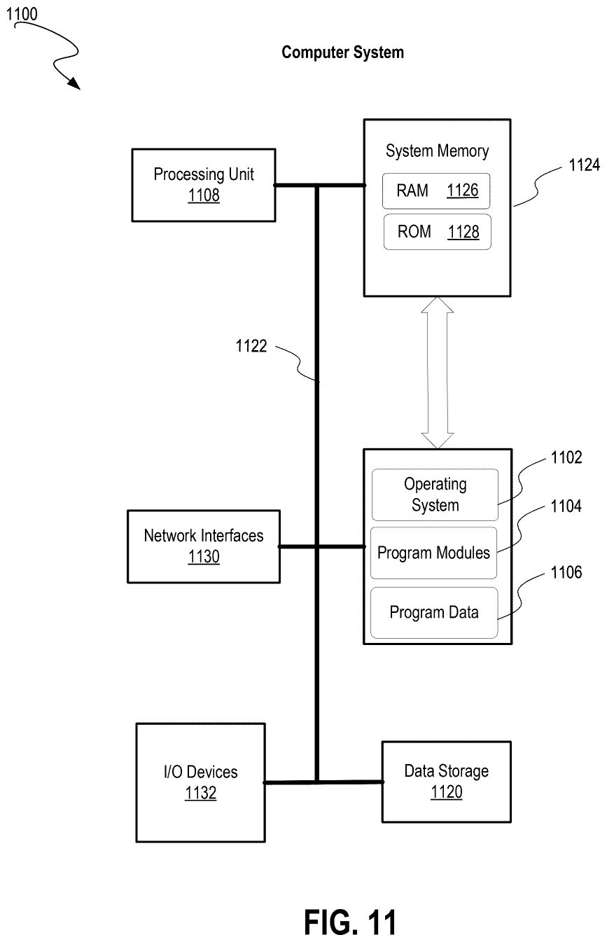

FIG. 11 illustrates a computer system 1100 to implement one or more methods of consultation between a service provider and a client, in accordance with at least one embodiment. Computer system 1100 comprises operating system 1102, program modules 1104, program data 1106, processing unit 1108, data storage 1120, system memory 1124 having random access memory (RAM) 1126 and/or read only memory (ROM) 1128, network interfaces 1130, I/O device 1132, and system bus 1122. Processing unit 1108 may include one or more processors (also referred to as microprocessors). Data storage 1120 may include a computer-readable storage medium (where the medium is any physical device or material on which data can be electronically and/or optically stored and retrieved). Computer readable storage medium/media also includes magnetic disks, optical disks, solid state drives, external memory systems, and flash memory drives. System bus 1122 provides an interface for system components including system memory 1124, to processing unit 1108. System bus 1122 can be any of several types of bus structure that can further interconnect to a memory bus (with or without controller), and a peripheral bus (e.g., PCI, PCIe, AGP, LPC, etc.), using any of a variety of commercially available bus architectures.

The techniques described herein such as extracting intent from the text or voice input, collecting electronic health records of a subject, preparing various versions of automated clinical notes, transcription and translation of the voice conversation during the consultation session, extracting condition of the subject from the input and EHR, preparing database of practitioners, suggesting suitable service provider based on the health state of a client, etc. can be implemented in computer-executable instructions (e.g., organized in program modules 1104). In at least one embodiment, program modules 1104 can include routines, programs, objects, components, and data structures that perform the tasks and implement data types for implementing the techniques described above. The functionality described herein can be performed, at least in part, by one or more hardware logic components.

In at least one embodiment, computer system 1100 can be one of several computers employed in a datacenter and/or computing resources (hardware and/or software) in support of cloud computing services for portable and/or mobile computing systems such as wireless communications devices, cellular telephones, and other mobile-capable devices. Cloud computing services, include infrastructure as a service, platform as a service, software as a service, storage as a service, desktop as a service, data as a service, security as a service, and APIs (application program interlaces) as a service. In some instances, system memory 1124 can include computer-readable storage (physical storage) medium such as a volatile memory (e.g. RAM 1126) and a non-volatile memory (e.g., ROM 1128). A basic Input/Output system (BIOS) can be stored in the non-volatile memory and includes the basic routines that facilitate the communication of data and signals between components within the computer system 1100, such as during startup. The volatile memory also includes high-speed RAM such as static RAM for caching data.

By way of example, and not limitation, system memory 1124 also may also include program modules 1104, which may include client applications, Web browsers, mid-tier applications, relational database management systems (RDBMS), etc., program data 1106, and an operating system 1102. By way of example, operating system 1102 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome OS, and the like) and/or mobile operating systems such as iOS, Windows® Phone, and Android OS operating systems. All or portions of operating system 1102, program modules 1104, and/or program data 1106 can also be cached in memory such as the volatile memory and/or non-volatile memory, for example (RAM 1126 or ROM 1128). The disclosed architecture can be implemented with current operating systems or combinations of operating systems (e.g., virtual machines).

In some other examples, the computer system 1100 may have additional features or functionality. For example, the computer system 1100 may also include additional data storage devices (removable and/or non-removable) such as, magnetic disks, optical disks, or tape. Computer-readable media may include, at least two types of computer-readable media, namely computer storage media and communication media. Computer storage media may include volatile and non-volatile, removable and non-removable media implemented in any method or technology for storage of information, such as computer readable instructions, data structures, program modules, or other data.

System memory 1124, and data storage 1120 including removable storage and non-removable storage are examples of computer storage media. Apart from RAM 1126 and ROM 1128, computer storage media includes, but is not limited to, EEPROM, flash memory or other memory technology, CD-ROM, digital versatile disks (DVD), or other optical storage, magnetic cassettes, magnetic tape, magnetic disk storage, or other magnetic storage devices, or any other non-transmission medium that can be used to store the targeted information and which can be accessed by computer system 1100. Moreover, the computer readable media may include computer-executable instructions that, when executed by the processing unit 1108, perform various functions and/or operations described herein. In contrast, communication media may embody computer readable instructions, data structures, program modules, or other data in a modulated data signal, such as a carrier wave, or other transmission mechanism.

Computer system 1100 may also include one or more input/output I/O devices 1132. One or more input devices of the one or more I/O devices 1132 may include keyboard, mouse, pen, voice input device, touch input device, etc., for example. The one or more output devices of the one or more I/O devices 1132 may be display, speakers, printers, etc., for example. These devices are well known in the art and are not discussed at length here. Computing system 1100 may also include one or more network interfaces 1130 to establish communication that may allow computer system 1100 to communicate with other system or devices, such as over a network. These networks may include wired networks as well as wireless networks. Here, computer system 1100 is one example of a suitable device or system and is not intended to suggest any limitation as to the scope of use or functionality of the various embodiments described.

Other well-known computer systems, environments and/or configurations that may be suitable for use with the embodiments include, but are not limited to personal computers, server computers, hand-held or laptop devices, multiprocessor systems, microprocessor-based systems, set top boxes, game consoles, programmable consumer electronics, network PCs, minicomputers, mainframe computers, or distributed computing environments that include any of the above systems or devices, and/or the like. In at least one embodiment, some or all the components of computer system 1100 are implemented in a cloud computing environment, such that resources and/or services are made available via a computer network for selective use by the user devices.

Although specific aspects have been described, various modifications, alterations, alternative constructions, and equivalents are possible. Embodiments are not restricted to operation within certain specific data processing environments but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components, or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

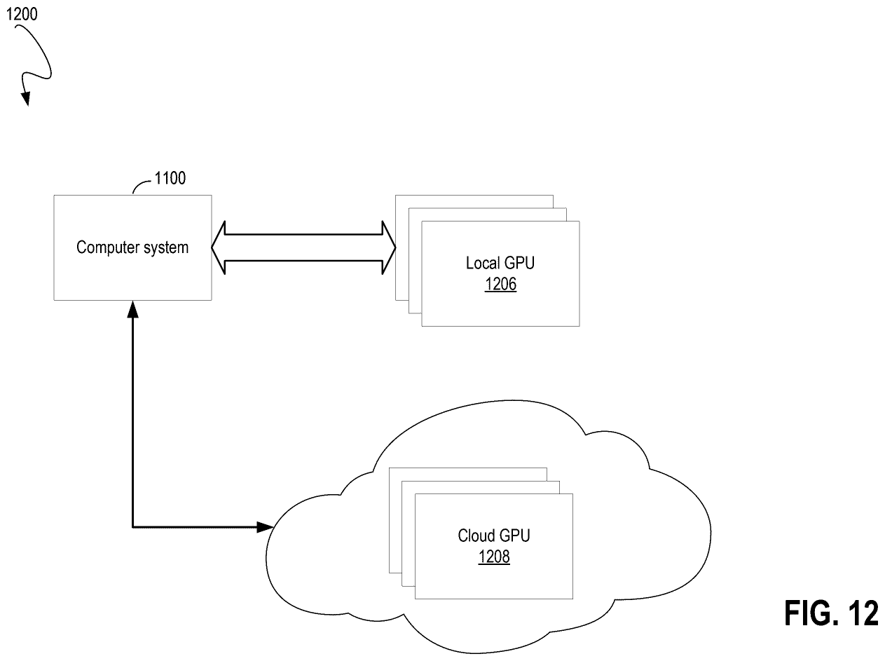

The computer system 1100 may have access to graphical processing units (GPU) for fast processing of the one or more machine-learning ML and deep learning methods as shown in FIG. 12. FIG. 12 illustrates a computer system having graphical processing units (GPUs) for execution of various machine learning methods, in accordance with at least one embodiment.

GPUs are specialized electronic circuits designed to accelerate certain types of compute workloads and change memory to accelerate creation of images in a frame buffer, intended for output to a display monitor. GPUs may be primarily used in rendering images, videos, and graphics-intensive applications like video games, 3D modeling, and scientific simulations. GPUs are highly suitable for running parallel processing tasks, making them much faster at rendering images and performing certain types of compute workloads compared to traditional central processing units (CPUs). Additionally, GPUs are increasingly being used for general-purpose computing tasks, such as machine learning and data processing to exploit their parallel computing capabilities.

GPUs enable AI based applications to handle large amounts of data and complex computations efficiently. Training deep neural networks, which are the backbone of many AI and ML models, involves processing large amounts of data through multiple layers of computations. GPUs excel at parallel processing, allowing them to train neural networks in a significantly smaller time compared with traditional CPUs. Frameworks like TensorFlow, PyTorch, and Keras leverage GPU acceleration to speed up the training process significantly. Once a neural network model is trained, GPUs can also enable faster inferences, predictions, or derive actional intelligence from the new data. GPUs may also accelerate inference tasks, enabling real-time or near-real-time performance in applications like image recognition, natural language processing, and recommendation systems. Many inference frameworks and libraries, such as NVIDIA's TensorRT, are optimized to take advantage of GPU hardware. Before training a neural network model, data may be preprocessed and augmented to optimize model performance and generalization. GPUs may accelerate these data processing tasks, such as image resizing, data normalization, and augmentation, allowing for faster data preparation pipelines. Tuning the hyperparameters of a machine learning model, such as learning rate, batch size, and regularization parameters, can be used for achieving good performance. GPUs may speed up hyperparameter optimization algorithms by parallelizing the evaluation of different parameter configurations, thereby enabling more efficient model tuning. GPUs may play a significant role in advancing deep learning research by enabling researchers to train and experiment with large scale language models, namely large language models (LLMs), more efficiently. The availability of powerful GPU clusters and cloud-based GPU services may democratize access to GPU resources, making it easier for researchers to conduct experiments and keep pushing the boundaries of AI and ML research. Integrated GPUs are built directly into a motherboard or processor and may commonly be found in consumer laptops, desktops, and entry-level systems. Integrated GPUs may share system memory (RAM), and may be less powerful than discrete GPUs but may be good for basic graphics tasks such as web browsing, video playback, video consultations, and light gaming, etc.

Discrete GPUs are separate graphics cards that may be installed in a computer system via PCIe slots. Discrete GPUs may have dedicated vRAM (Video Random Access Memory) and may be more powerful than integrated GPUs, making them suitable for compute intensive tasks such as gaming, content creation, and professional applications like 3D modeling and rendering. Workstation GPUs may be optimized for professional applications such as CAD (Computer-Aided Design), CAM (Computer-Aided Manufacturing), CGI (Computer-Generated Imagery), and scientific simulations. Workstation GPUs may offer features like ECC (Error-Correcting Code) memory and certified drivers for stability and reliability in professional workflows; for example, NVIDIA's Quadro series and AMD's Radeon Pro series etc. Data center GPUs are designed for high-performance computing (HPC), deep learning, and AI workloads in data centers and cloud environments like NVIDIA's Tesla and AMD's Instinct series, etc. Data center GPUs may feature specialized hardware and software optimizations for parallel processing and AI based accelerations. Mobile GPUs are designed for use in smartphones, tablets, and other portable devices. Mobile GPUs may prioritize power efficiency and thermal management to deliver acceptable performance within the constraints of mobile devices. Mobile GPUs may include, for example, Qualcomm's Adreno GPUs (used in Snapdragon SoCs), ARM's Mali GPUs, and Apple's custom GPUs (used in iPhones and iPads), etc.

Some systems may have a single GPU, while others, especially those used for high-performance computing or deep learning, may have multiple GPUs. Some systems may use technologies like NVIDIA's NVLink or AMD's Infinity Fabric to enable high-speed communication between GPUs. GPUs may generate a significant amount of heat, especially under heavy workloads. Proper cooling solutions, such as fans or liquid cooling systems, may be used to maintain good performance and prevent overheating. Additionally, GPUs may require adequate power supply. Configuring GPU drivers and optimizing software settings may also impact performance. This may include installing the appropriate drivers for the GPU model and adjusting settings within software applications to take advantage of GPU acceleration when available. GPU drivers are software components that may allow the operating system to communicate with the GPU hardware. GPU drivers may be provided by the GPU manufacturers, such as NVIDIA or AMD, and can be installed and updated regularly to achieve good performance and compatibility with the new applications and games. Graphics APIs like DirectX (Microsoft), OpenGL, Vulkan, and Metal (Apple) may provide a standardized way for software developers to interact with the GPU hardware. These APIs enable developers to create graphics-intensive applications, including video games, 3D modeling software, multimedia applications, and machine learning methods.

GPU monitoring and overclocking tools may allow users to monitor the temperature, usage, and performance of GPUs in real-time. GPU tools may also provide features for overclocking, which involves increasing the clock speeds of the GPU to achieve higher performance. Popular GPU monitoring and overclocking tools include MSI Afterburner, EVGA Precision X1, and GPU-Z. GPUs are increasingly being used for general-purpose computing tasks, including machine learning, scientific simulations, and data processing. GPU computing libraries and frameworks like CUDA (NVIDIA), OpenCL, and TensorFlow may provide developers with the tools and APIs to leverage the parallel processing capabilities of GPUs for these tasks. Many software applications leverage GPU hardware to accelerate tasks, such as video editing, image processing, and 3D rendering. These applications may be optimized to offload specific computations to the GPU, resulting in improved performance and faster processing times. Core Clock Speed is the base operating frequency of a GPU core and may be the primary factor in determining the overall performance of the GPU. Modern GPUs may also feature a boost clock speed, which is a higher frequency that the GPU may dynamically achieve under conditions such as availability of additional thermal headroom or when the workload demands increased performance. The boost clock speed may allow the GPU to temporarily operate at a higher frequency and may deliver better performance in demanding applications.

GPUs may have memory specifically designed for handling graphical data and computations efficiently. This memory may be used for storing textures, shaders, geometry data, frame buffers, and other resources used in rendering graphics. The memory on GPUs may serve as a high-speed intermediary between the CPU and the GPU, facilitating quick data transfers and computations, which may be vital for rendering high-resolution graphics and running complex simulations. Graphics double data rate (GDDR) memory is specifically optimized for GPUs and may be used in graphics cards. GDDR memory may come in different versions, such as GDDR5, GDDR5X, GDDR6, and GDDR6X. High bandwidth memory (HBM) may provide higher bandwidth and lower power consumption compared to traditional GDDR memory. Memory chips may be placed on a stacked configuration in HBM, allowing for a significant increase in memory bandwidth while reducing the physical footprint on the GPU. Some GPUs may feature shared memory, which may be accessible by both the GPU and the CPU. This memory may be used for communication between the CPU and GPU and for storing data to be accessed by both processing units. The amount of memory on a GPU may vary depending on the specific model and intended use case. Professional-grade GPUs used for tasks like rendering, machine learning, and scientific simulations may have memory ranging from 8 GB to 48 GB or beyond.

Cloud-based GPUs, also known as GPU instances or GPU cloud services, may offer access to powerful graphics processing units (GPUs) hosted in remote data centers. These services may allow users to rent GPU resources on-demand while not investing in physical hardware or infrastructure. Cloud-based GPU instances may also be utilized for AI and machine learning applications. Cloud-based GPUs may provide the computational power to train large-scale deep learning models, known as LLMs, and perform inference on real-time data. Frameworks like TensorFlow, PyTorch, and MXNet may often be used in conjunction with cloud-based GPU instances for AI and ML workloads. High-performance computing (HPC) applications, such as scientific simulations, weather forecasting, and computational fluid dynamics may utilize cloud-based GPU instances to leverage the parallel processing capabilities to perform these computationally intensive tasks. Cloud-based GPUs may be used in the media and entertainment industry for rendering 3D graphics, visual effects, and animation. Rendering tasks that would otherwise require expensive on-premises hardware may be offloaded to cloud-based GPU instances, allowing for faster rendering times and increased scalability. Cloud gaming platforms may leverage cloud-based GPU instances to stream high-quality video games to users' devices over the internet. These platforms may handle the rendering and processing of games on remote servers, enabling users to play complex games on low-powered devices such as smartphones, tablets, and smart TVs etc. Cloud-based GPUs may be used for processing and analyzing large datasets in fields such as data science, analytics, and visualization. GPU-accelerated databases and analytics platforms may leverage the parallel processing power of GPUs to accelerate queries, data processing, and visualization tasks. Leading cloud service providers such as Amazon Web Services (AWS), Microsoft Azure, Google Cloud Platform (GCP), and NVIDIA may offer a variety of GPU instances tailored to different use cases and workloads. These instances may come with different GPU types, such as NVIDIA Tesla GPUs, varying amounts of GPU memory, and options for CPU configurations.

In at least one embodiment, the computer system 1100 in FIG. 12 has access to one or more local GPUs 1206. The one or more GPUs 1206 may come from manufacturers, such as NVIDIA®, AMD®, Intel®, ARM®, or Qualcomm® etc. In at least one embodiment, the GPU cloud services over the internet are utilized to obtain GPU functionality. Cloud GPU 1208 may be provided, for example, by Amazon® Elastic Compute Cloud (EC2), Google® Cloud Platform (GCP) Compute Engine service, Microsoft® Azure Virtual Machines service, Oracle® Cloud Infrastructure (OCI) Compute service, IBM® Cloud Virtual Servers service, etc.

Specific details are given in this disclosure to provide a thorough understanding of the aspects. However, aspects may be practiced without these specific details. For example, well-known circuits, processes, algorithms, structures, and techniques have been shown without unnecessary detail to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

The following examples are provided that illustrate the various examples of the disclosure. The examples can be combined with other examples. As such, various examples can be combined with other examples without changing the scope of the invention.

Example 1 is a computer-implemented method comprising: receiving an electronic input from one or more service providers that are located worldwide, wherein the electronic input includes a set of credentials associated with the one or more service providers, wherein the set of credentials include one or more of personal data, professional license, educational degrees, one or more certificates, and work history; verifying the one or more service providers based on the set of credentials that are validated from one or more regulatory authorities including licensing authority, certification authority, and professional association; preparing a database of service providers by including the one or more service providers that are verified into the database; implementing an access control mechanism based on a user's role, permission, and authentication credential to prevent unauthorized access from viewing or modifying the database; and employing an encryption protocol to encrypt the database at rest and in transit.

Example 2 is a computer-implemented method as in any of the examples, particularly example 1, further including: receiving an input from a client, wherein the input includes a client reservation; accessing a set of attributes associated with the client from one or more electronic records, wherein the set of attributes include one or more of client personal information, demographics, client preferences, insurance policy and geographic location; applying one or more natural language processing (NLP) models configured to extract the client reservation from the input; identifying, based on the client reservation and the set of attributes, a subset of service providers from the database of service providers; selecting a service provider from the subset of service providers based on the set of attributes and client reservation; and scheduling an appointment of the client with the selected service provider for a video conferencing session.

Example 3 is a computer-implemented method as in any of the examples, particularly example 2, further including: establishing a secure network connection between the selected service provider and the client as a video conference session at the appointment; and sharing data of the client to the selected service provider during the video conference session.

Example 4 is a computer-implemented method as in any of the examples, particularly example 3, further including: receiving a first preferred language input from the client and a second preferred language input from the selected service provider; detecting, in real time, a first speaking language from a client speech of the client and a second speaking language from a service provider speech of the selected service provider; translating, in real-time during the video conference session by leveraging one or more natural language processing models: the client speech from the first speaking language to the second preferred language; and the service provider speech from the second speaking language to the first preferred language; outputting, in real-time the translated client speech to an audio interface of the selected service provider; and outputting in real-time the translated service provider speech to the audio interface of the client.

Example 5 is a computer-implemented method as in any of the examples, particularly example 4, further including: transcribing in real-time, the client speech and the service provider speech during the video conferencing session by leveraging one or more speech recognition models; extracting information from the transcribed client speech and the transcribed service provider speech by applying the one or more natural language processing models; accessing one or more parameters associated with the extracted information from the one or more electronic records of the client to contextualize the client speech and the service provider speech; generating in real-time, clinical notes based on the one or more parameters; and updating the one or more parameters in the one or more electronic records of the client.

Example 6 is a system comprising: one or more processors; and a non-transitory computer-readable storage medium containing instructions which, when executed on the one or more processors, cause the one or more processors to perform a method including: receiving an electronic input from one or more service providers located anywhere worldwide, wherein the electronic input includes a set of professional credentials associated with the one or more service providers, wherein the set of professional credentials include one or more of personal data, professional license, educational degrees, certificates, and work history; verifying the one or more service providers based on the set of professional credentials that are validated from one or more regulatory authorities including licensing boards, certification bodies, and/or professional associations; and preparing a database of service providers by including the one or more service providers that are verified.

Example 7 is a system as in any of the examples, particularly example 6, wherein the method further includes: receiving an input from a subject, wherein the input includes a problem condition; accessing a set of attributes associated with a client from one or more electronic records, wherein the set of attributes include one or more of subject personal information, subject demographics, subject preferences, insurance policy and geographic location; applying one or more machine-learning (ML) models configured to extract the problem condition from the input; identifying, based on the problem condition and the set of attributes, a subset of service providers from the database of service providers; selecting a service provider from the subset of service providers based on the subject preferences, set of attributes, and problem condition; and scheduling an appointment of the subject with the selected service provider for a video conferencing session.

Example 8 is a system as in any of the examples, particularly example 7, wherein the method further includes: establishing a secure connection between the selected service provider and the subject as a video conference session is initiated at the appointment; and providing the client and the selected service provider to share data during the video conference session.

Example 9 is a system as in any of the examples, particularly example 7, wherein the method further includes: receiving a first preferred language input from a subject; receiving a second preferred language input from the selected service provider; detecting the first preferred language input and the second preferred language input; transcribing, in real-time, a first speech during the video conferencing session by leveraging one or more speech recognition models in a second preferred language; transcribing, in real-time, a second speech during the video conferencing session by leveraging one or more speech recognition models in a first preferred language; applying a second one or more machine-learning models configured to extract problem information from the transcribed first speech and the transcribed second speech; accessing one or more parameters associated with the problem information from an electronic health record (EHR) of the subject to contextualize the first speech and second speech with a history of the subject; generating, in real-time, clinical notes based on the one or more parameters; and updating the one or more parameters in the EHR of the subject.

Example 10 is a system as in any of the examples, particularly example 6, which further includes: processing the input via one or more natural language processing models to extract an intent of the input.

Example 11 is a system as in any of the examples, particularly example 6, which further includes providing data encryption for data at rest and at transit.

Example 12 is a computer-program product tangibly embodied in a non-transitory machine-readable storage medium, including instructions configured to cause one or more data processors to perform a method including: receiving an electronic input from one or more service providers, wherein the electronic input includes a set of professional credentials associated with the one or more service providers located anywhere worldwide, wherein the set of professional credentials include one or more of personal data, professional license, educational degrees, certificates, and work history; verifying the one or more service providers based on the set of professional credentials that are validated from one or more regulatory authorities including licensing boards, certification bodies, and/or professional associations; and preparing a database of service providers by including the one or more service providers that are verified.

Example 13 is a computer-program product as in any of the examples, particularly example 12, wherein the method further includes: receiving an input from a subject, wherein the input includes a problem condition; accessing a set of attributes associated with a client from one or more electronic records, wherein the set of attributes include one or more of subject personal information, subject demographics, subject preferences, insurance policy, and geographic location; applying one or more machine-learning (ML) models configured to extract the problem condition from the input; identifying, based on the problem condition and the set of attributes, a subset of service providers from the database of the one or more service providers; selecting a service provider from the subset of service providers based on the subject preferences, set of attributes, and problem condition; and scheduling an appointment of the subject with the selected service provider for a video conferencing session.

Example 14 computer-program product of example 13, wherein the method further includes: establishing a secure connection between the selected service provider and a subject as a video conference session is initiated at the appointment; and providing the client and the selected service provider to share data during the video conference session.

Example 15 is a computer-program product of example 14, wherein the method further includes: receiving a first preferred language input from the subject; receiving a second preferred language input from the selected service provider; detecting the first preferred language input and the second preferred language input; transcribing, in real-time, a first speech during the video conferencing session by leveraging one or more speech recognition models in a second preferred language; transcribing, in real-time, a second speech during the video conferencing session by leveraging one or more speech recognition models in a first preferred language; applying a second one or more machine-learning models configured to extract problem information from the transcribed first speech and the transcribed second speech; accessing one or more parameters associated with the problem information from an electronic health record (EHR) of the subject to contextualize the first speech and second speech with a history of the subject; generating, in real-time, clinical notes based on the one or more parameters; and updating the one or more parameters in the EHR of the subject.

Example 16 is a computer-program product of example 12, wherein the method further includes processing the input via one or more natural language processing models to extract an intent of the input.

Example 17 is a computer-program product of example 12, wherein the method further includes providing data encryption for data at rest and at transit.

What is claimed is:

1. A computer-implemented method comprising:
receiving an electronic input from one or more service providers that are located worldwide, wherein the electronic input includes a set of credentials associated with the one or more service providers, wherein the set of credentials include one or more of personal data, professional license, educational degrees, one or more certificates, and work history;
verifying the one or more service providers based on the set of credentials that are validated from one or more regulatory authorities including licensing authority, certification authority, and professional association;
preparing a database of service providers by including the one or more service providers that are verified into the database;
implementing an access control mechanism based on a user's role, permission, and authentication credential to prevent unauthorized access from viewing or modifying the database; and
employing an encryption protocol to encrypt the database at rest and in transit.

2. The computer-implemented method of claim 1, further including:
receiving an input from a client, wherein the input includes a client reservation;
accessing a set of attributes associated with the client from one or more electronic records, wherein the set of attributes include one or more of client personal information, demographics, client preferences, insurance policy, and geographic location;
applying one or more natural language processing (NLP) models configured to extract the client reservation from the input;
identifying, based on the client reservation and the set of attributes, a subset of service providers from the database of service providers;
selecting a service provider from the subset of service providers based on the set of attributes and client reservation; and
scheduling an appointment of the client with the selected service provider for a video conferencing session.

3. The computer-implemented method of claim 2, further including:
establishing a secure network connection between the selected service provider and the client as a video conference session at the appointment; and
sharing data of the client to the selected service provider during the video conference session.

4. The computer-implemented method of claim 3, further including:
receiving a first preferred language input from the client and a second preferred language input from the selected service provider;
detecting, in real time, a first speaking language from a client speech of the client and a second speaking language from a service provider speech of the selected service provider;
translating, in real-time, during the video conference session by leveraging one or more natural language processing models:
the client speech from the first speaking language to the second preferred language; and
the service provider speech from the second speaking language to the first preferred language;
outputting, in real-time, the translated client speech to an audio interface of the selected service provider; and
outputting, in real-time, the translated service provider speech to the audio interface of the client.

5. The computer-implemented method of claim 4, further including:
transcribing, in real-time the client speech and the service provider speech during the video conferencing session by leveraging one or more speech recognition models;
extracting information from the transcribed client speech and the transcribed service provider speech by applying the one or more natural language processing models;
accessing one or more parameters associated with the extracted information from the one or more electronic records of the client to contextualize the client speech and the service provider speech;
generating, in real-time, clinical notes based on the one or more parameters; and
updating the one or more parameters in the one or more electronic records of the client.

6. A system comprising:
one or more processors; and
a non-transitory computer-readable storage medium containing instructions which, when executed on the one or more processors, cause the one or more processors to perform a method including:
receiving an electronic input from one or more service providers located anywhere worldwide, wherein the electronic input includes a set of professional credentials associated with the one or more service providers, wherein the set of professional credentials include one or more of personal data, professional license, educational degrees, certificates, and work history;
verifying the one or more service providers based on the set of professional credentials that are validated from one or more regulatory authorities including licensing boards, certification bodies, and/or professional associations; and
preparing a database of service providers by including the one or more service providers that are verified.

7. The system of claim 6, wherein the method further includes:
receiving an input from a subject, wherein the input includes a problem condition;
accessing a set of attributes associated with a client from one or more electronic records, wherein the set of attributes include one or more of subject personal information, subject demographics, subject preferences, insurance policy, and geographic location;
applying one or more machine-learning (ML) models configured to extract the problem condition from the input;
identifying, based on the problem condition and the set of attributes, a subset of service providers from the database of service providers;
selecting a service provider from the subset of service providers based on the subject preferences, set of attributes, and problem condition; and
scheduling an appointment of the subject with the selected service provider for a video conferencing session.

8. The system of claim 7, wherein the method further includes:
establishing a secure connection between the selected service provider and the subject as a video conference session is initiated at the appointment; and
providing the client and the selected service provider to share data during the video conference session.

9. The system of claim 7, wherein the method further includes:
receiving a first preferred language input from a subject;
receiving a second preferred language input from the selected service provider;
detecting the first preferred language input and the second preferred language input;
transcribing, in real-time, a first speech during the video conferencing session by leveraging one or more speech recognition models in a second preferred language;
transcribing, in real-time, a second speech during the video conferencing session by leveraging one or more speech recognition models in a first preferred language;
applying a second one or more machine-learning model configured to extract problem information from the transcribed first speech and the transcribed second speech;
accessing one or more parameters associated with the problem information from an electronic health record (EHR) of the subject to contextualize the first speech and second speech with a history of the subject;
generating, in real-time, clinical notes based on the one or more parameters; and
updating the one or more parameters in the EHR of the subject.

10. The system of claim 6, further includes: processing the input via one or more natural language processing models to extract an intent of the input.

11. The system of claim 6, further includes providing data encryption for data at rest and at transit.

12. A computer-program product tangibly embodied in a non-transitory machine-readable storage medium, including instructions configured to cause one or more data processors to perform a method including:
receiving an electronic input from one or more service providers, wherein the electronic input includes a set of professional credentials associated with the one or more service providers located anywhere worldwide, wherein the set of professional credentials include one or more of personal data, professional license, educational degrees, certificates, and work history;
verifying the one or more service providers based on the set of professional credentials that are validated from one or more regulatory authorities including licensing boards, certification bodies, and/or professional associations; and
preparing a database of service providers by including the one or more service providers that are verified.

13. The computer-program product of claim 12, wherein the method further includes:
receiving an input from a subject, wherein the input includes a problem condition;
accessing a set of attributes associated with a client from one or more electronic records, wherein the set of attributes include one or more of subject personal information, subject demographics, subject preferences, insurance policy, and geographic location;
applying one or more machine-learning (ML) models configured to extract the problem condition from the input;
identifying, based on the problem condition and the set of attributes, a subset of service providers from the database of the one or more service providers;
selecting a service provider from the subset of service providers based on the subject preferences, set of attributes, and problem condition; and
scheduling an appointment of the subject with the selected service provider for a video conferencing session.

14. The computer-program product of claim 13, wherein the method further includes:
establishing a secure connection between the selected service provider and a subject as a video conference session is initiated at the appointment; and
providing the client and the selected service provider to share data during the video conference session.

15. The computer-program product of claim 14, wherein the method further includes:
receiving a first preferred language input from the subject;
receiving a second preferred language input from the selected service provider;
detecting the first preferred language input and the second preferred language input;
transcribing, in real-time, a first speech during the video conferencing session by leveraging one or more speech recognition models in a second preferred language;
transcribing, in real-time, a second speech during the video conferencing session by leveraging one or more speech recognition models in a first preferred language;
applying a second one or more machine-learning models configured to extract problem information from the transcribed first speech and the transcribed second speech;
accessing one or more parameters associated with the problem information from an electronic health record (EHR) of the subject to contextualize the first speech and second speech with a history of the subject;
generating, in real-time, clinical notes based on the one or more parameters; and
updating the one or more parameters in the EHR of the subject.

16. The computer-program product of claim 12, wherein the method further includes processing the input via one or more natural language processing models to extract an intent of the input.

17. The computer-program product of claim 12, wherein the method further includes providing data encryption for data at rest and at transit.
